(19) United States
(12) Patent Application Publication
(10) Pub. No.: US 2018/0317769 A1
(43) Pub. Date: Nov. 8, 2018

(54) SYSTEM AND METHOD FOR REAL TIME BLOOD GLUCOSE MONITORING AND RESPONSE

(71) Applicant: Sajid Khan, New Hyde Park, NY (US)

(72) Inventor: Sajid Khan, New Hyde Park, NY (US)

(21) Appl. No.: 15/588,948

(22) Filed: May 8, 2017

(51) Int. Cl. A61B 5/00; A61B 5/145

(52) U.S. Cl. CPC A61B 5/0004; A61B 5/14532; A61B 5/7221; A61B 5/7282; G06F 19/3418; A61B 5/746; A61B 5/747; A61B 5/0022; A61B 5/743

(57) ABSTRACT

A system for remotely capturing and processing daily blood glucose data and of a patient comprises a first device configured to input blood glucose data of a patient, wherein the data is validated and encrypted within the first device. The First device is in wireless communication with the Second device, wherein the validated blood glucose level data should be at least for a concentration of blood glucose, and wherein the analysis data reading(s) of the blood glucose is transmitted to the database on Second device, wherein the database stores the analysis. A second device receives the validated data readings of the blood glucose, and wherein the blood glucose data reading(s) is combined with one or more previous data reading(s) already stored on the system.

### CROSS-REFERENCE TO RELATED APPLICATION(S)

Not applicable

### BACKGROUND OF THE INVENTION

### 1. Field of Invention

The present invention relates to the field of physiological, medical, and biochemical processes analysis. Specifically to the real-time evaluation of patient blood glucose levels.

### 2. Description of Related Art

The human body provides for a number of mechanisms and systems for regulating homeostasis. Of these systems, storage of macro or micro nutrients involves a number of body systems and organs producing, receiving, and reacting to various hormones. One of the most important functions for these systems is the handling, including storage and use, of glucose.

The human body requires glucose for cellular operation including energy. Glucose is derived through ingestion and, when applicable, subsequent breakdown of complex sugars and molecules to the ultimate component of glucose which is the form capable of being metabolized by cells for energy.

Insulin is the hormone released by the pancreas and serves to relay messages to cells instructing them to uptake glucose from the bloodstream. Where there is a demand for energy and low blood glucose levels, insulin can serve to convert stored glycogen making up for any deficiency.

Glucagon is a hormone working in opposition to insulin and instructs the storage of excess glucose resulting from ingested foods. Through multitude of pathways involving negative feedback loops, the body signals and provides instructions for use and/or storage of glucose. During normal operation, glucose can be stored in the liver and muscles as glycogen to be used later between meals or at times of fasting.

Where there has been damage or disease resulting in damage to the pancreas or genetic disorders resulting in a malfunction of insulin operation, a condition exists referred to a diabetes. Diabetes is generally split into two main types: Type I and Type II. Type I diabetes is generally characterized by an auto-immune response whereby the body's own antibodies work to inhibit insulin production and/or release. This is a type of diabetes whereby a patient is insulin-dependent for external sources of insulin to regulate blood glucose levels. Type II diabetes is characterized by external factors such as obesity resulting in a lack of insulin production not enough to meet the body's need.

Diagnosis and management of diabetes relies on constant measurement and analysis of the body's blood glucose levels. Current technology provides for a number of ways someone with diabetes can determine their blood glucose in real-time through the use of handheld components and a finger-stick method to produce a sample of blood for analysis within the handheld component.

After the levels are identified, the patient is responsible for manually recording outputs to discuss with their treating physician at a later time. Such manual interaction obviously presents a number of issues including lack of accurate recording, lack of consistent recording, lack or historical trends, speculation in the functionality of the testing overall, and a lack of real0time communication to the treating physician. As with any procedure requiring the manual recording of data, human error may result in catastrophically negative results including serious injury or death.

Based on the foregoing, there is a need in the art for a system that will allow of automatic and autonomous recording or blood glucose data received during finger stick or other methods. A system is needed providing for a method and process of the real-time trend creation, analysis management, and information transfer to individuals responsible for monitoring and treatment regiment establishment.

### SUMMARY OF THE INVENTION

This invention includes, methods and software systems user interface for manual or automated input of Glycemic data installed and configured on wireless enabled communication device—herein after known as the First device. The first device also has additional modules to encrypt and automatically transmit patients' Glycemic data in a contact less manner and in real-time to the remote host computer—herein after known as the Second device, to store, automatically process and encrypt the stored data. A configurable workflow engine software module installed on the Second device to automatically distribute/publish the Glycemic data to end user systems for use by Healthcare staff, clinicians or research faculties for managing patient's health condition or research purposes.

In an embodiment, a user engages the First device, wherein the user inputs one or more data into the First device, wherein the one or more data includes at least a threshold value of the concentration of blood glucose- the Glycemic data.

The invention along with the methods and software can capture, validate, encrypt and transmit the patient data remotely and in secure manner so that the paper maintenance of such data is not required by the patient to record the required Glycemic data. This eliminates the need for the patient to manage any data on paper as well as to visit medical facility or Healthcare staff to visit patient to present or obtain such data in physical format, resulting in patient convenience, remote patient Glycemic level monitoring and timely medical intervention, time & cost savings for both parties. This data can then be used by Healthcare staff for any specialty for patient assessment, routine patient monitoring, pre & post care for/after a surgical or non-surgical procedure, medical condition studies, drug or dosage optimization and or real-time analysis of patients' condition and symptoms. Additionally the invention also eliminates manual data capture and data entry mistakes, prevents the loss of Healthcare staff time from missed patient appointments, provides a method of continuous patient health monitoring and ensures timely data availability for clinical and reporting procedures. Additionally the data capture and communication software on First Device or on a Second Device can be configured to transmit or share the patient data to other available electronic data devices or health information management systems. This data integration with other available healthcare systems for making patient healthcare and diagnosis more accurate and easier.

In an embodiment, the patient is authenticated, wherein one or more unique medical record identifiers are provided, wherein the one or more unique medical record identifiers are associated with the patient, and wherein when the one or more unique medical record identifiers are accepted, a confirmation is received by the first from the second device.

In an embodiment, the analysis of the Blood Glucose data is cross-referenced against the Glycemic status thresholds, wherein the Glycemic thresholds are predetermined based on the Industry Standards, and wherein a level of Glycemic status is identified, wherein the Glycemic level corresponds to the blood glucose concentration.

In an embodiment, if the level of Glycemic status deviates from a predetermined threshold level, an alert is provided to at least the first device and the user interface on Second device.

In an embodiment, a display provides visual representation of the analysis and the level of Glycemic status.

In an embodiment, the visual representation comprises output patient glucose, insulin charts, advisory messages, and patient alerts based on the concentration of blood glucose.

In an embodiment, the database stores one or more medications prescribed to the patient.

In an embodiment, a physician module within the second device is configured to alter medication type and medication dosage.

In an embodiment, the system further comprises a telecommunications module configured to communicate patient status data to an emergency responder.

In an embodiment, the system further comprises a data provisioning module to upload data to a third party system.

In an embodiment, the first device is a cellular phone or any mobile data communication device.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a flow diagram view of the system operation, according to an embodiment of the present invention;

FIG. 2 is a flow diagram view of the system operation, according to an embodiment of the present invention;

FIG. 3 is a flow diagram view of the system operation, according to an embodiment of the present invention;

FIG. 4 is a flow diagram view of the system operation, according to an embodiment of the present invention;

### DETAILED DESCRIPTION OF PREFERRED EMBODIMENTS

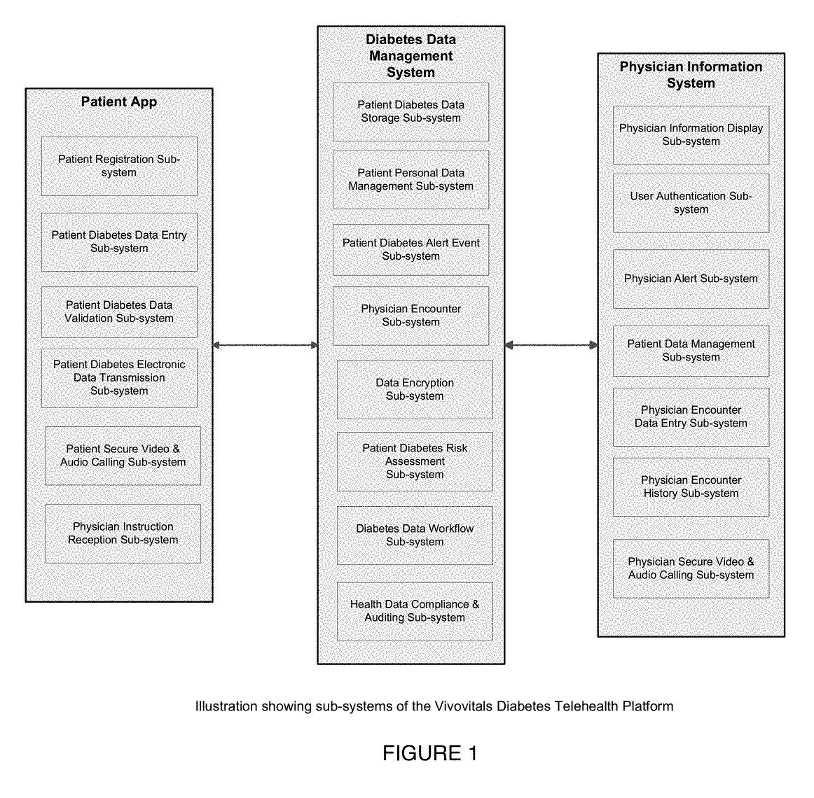

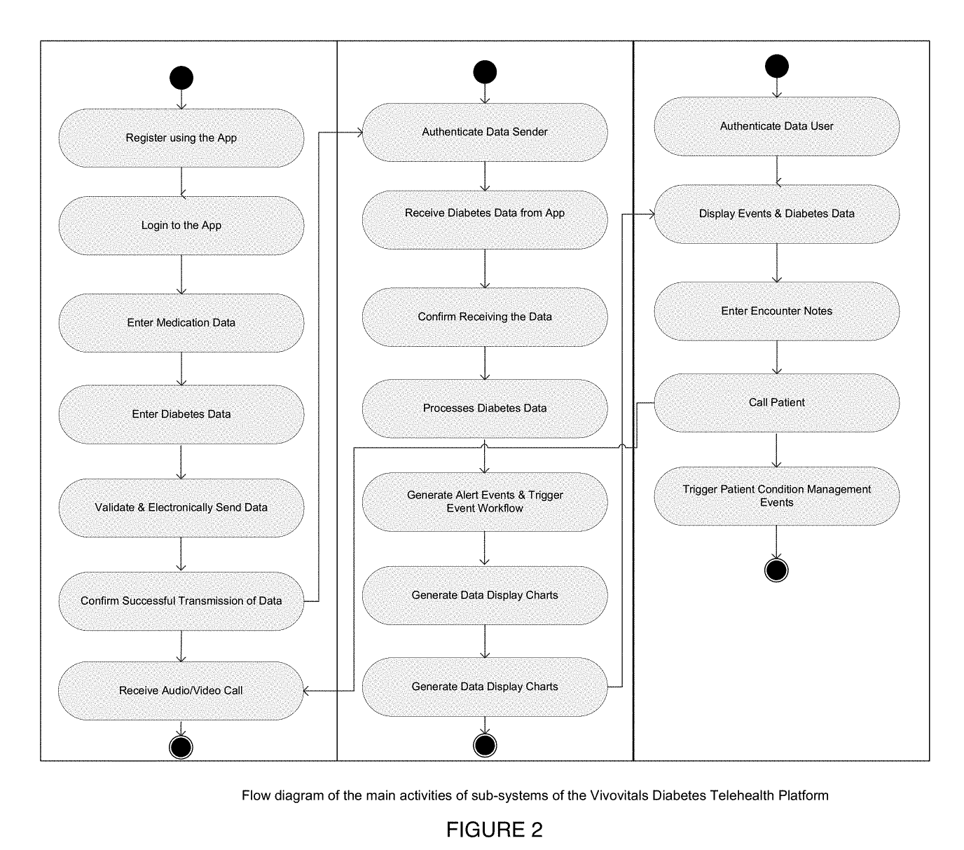

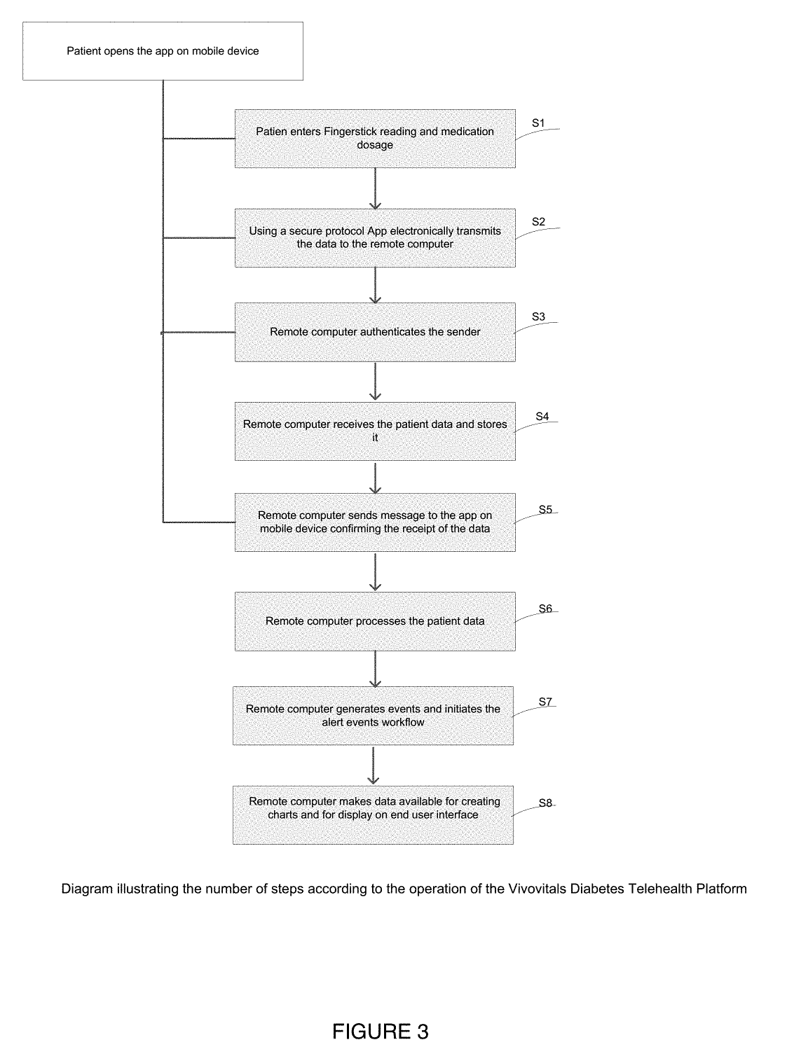

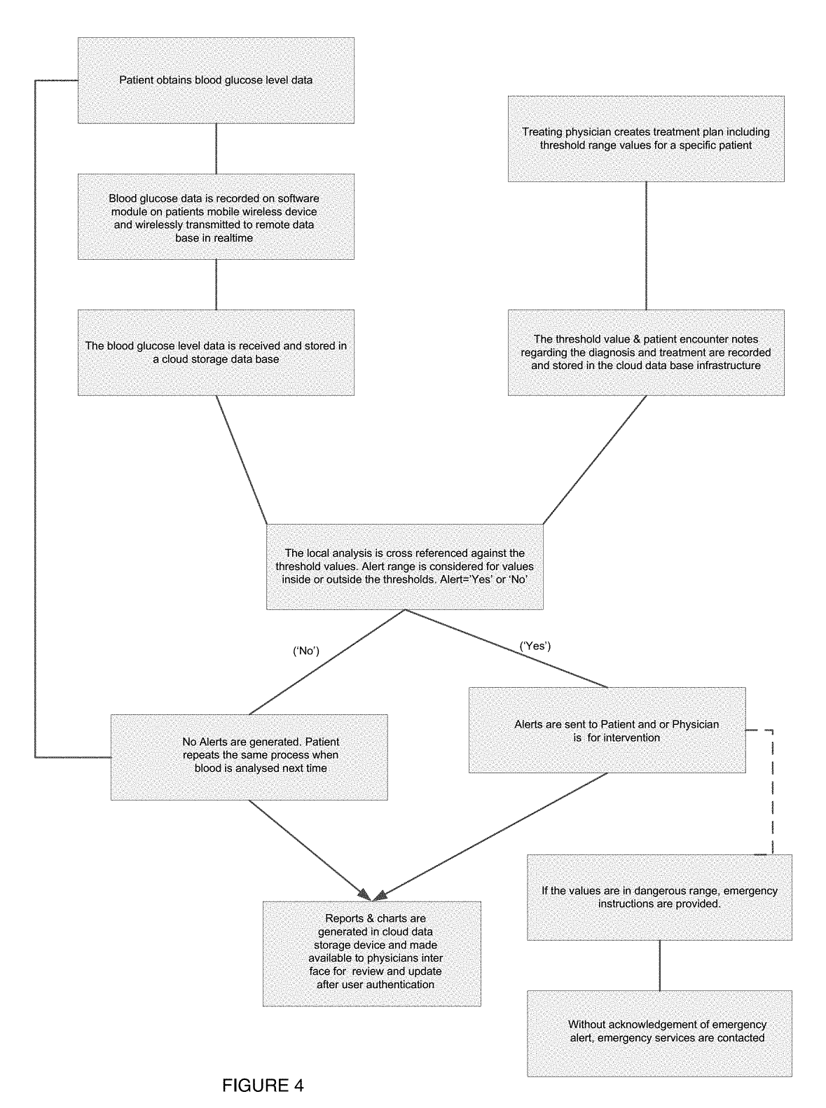

Preferred embodiments of the present invention and their advantages may be understood by referring to FIGS. 1-4, wherein like reference numerals refer to like elements.

A system and method for real-time blood glucose (BG) data capture and realtime transmission has multiple steps including a data entry module on a wireless device. The obtained data is validated by the software module on the device. The data is encrypted and then transmitted over one or more networks to a central database where the information is stored.

The present system essentially provides for a digital finger stick blood sugar log book data capture and transmission in real time from patient's device to a second system(s) which is used by doctor with healthcare management user interface module. The data can be viewed and accessed from any digital device.

The system also provides for automatic/instant response alerts and recommendations from physician via. the end user interface and is transmitted electronically back to the patient mobile device for any action regarding blood sugar levels and suggestions for appropriate intervention(s). These alerts can be automatically setup based on standardized norms or specifically incorporated based on the patient and treating physicians understanding of the patient's condition.

The present system eliminates the patient's requirement to carry a paper log book while maintaining history of sample readouts or when visiting a physician.

The present system improves the quality of diabetes management and patient's health in between doctor's visits by continuous communication between patient and doctor via digital real time finger stick blood sugar numbers and alerts suggestions through a system to maintain endo-telehealth consultation history log.

The present system also automatically calculates, generates and submits physician Telehealth consultation invoice for payments to the payer electronically. The graphical user interface may provide for options relating to completion of services and methods for payment for such services.

The present system allows for the creation, customization and maintenance of healthcare encounter templates for physicians and maintains EMR history of all such encounters .

The system incorporates a TeleHealth analytical module, which is based on daily physiological and psychological measures to assess patient specific treatment needs, feed this into the personalized treatment design, and then gather appropriate evidence to enable physicians to make decisions.

A patient Care Information Portal within the system helps patients understand their long-term condition and cope better with the management of their disease.

The system also incorporates a module to measure blood sugar levels without pricking the fingers and submit the readings to physicians using mobile device.

The information may include relevant patient data for identification purposes. For example, the information transmitted and stored within the database may include the patient's name, treating physician, conditions being managed, and specific threshold values of blood glucose levels which are predetermined by the treating physician.

A graphical user interface (GUI) is presented on a on-premise or mobile computing device for user interaction. The GUI provides for a platform by which the treating physician and/or the patient are able to review information stored within the database. In engaging the GUI, necessary verification and identification protocols are followed to access and assurance accurate review of a particular patient. The GUI platform serves to request data from the database after verification has been achieved. Identification of the patient is input through the GUI and transmitted across the one or more networks. A processor serves to identify the specifically requested patient information within the database based on the request generated through the GUI. Once the information is identified on the database, the information is transmitted to the local portal presenting the GUI where the information request was established. The information is then displayed for consideration by the treating physician.

Levels of verification include procedures and protocols observed by the medical community and professionals therein. For example, legal-based procedures such as Health Insurance Portability and Accountability (HIPAA) dictate certain standards and practices when handling and transmitting patient information.

The information displayed to the treating physician is presented on a screen providing for a number of options and templates to engage the data transmitted from the database. For example, certain options may exist whereby the treating physician can identify a specific day/time range of data recovered from the BG meter (previously transmitted to the database). Other options allow the treating physician to instruct the computing device to develop graphical representations of the data illustrating trends and visually identifying outliers or anomalies.

The treating physician may have a specific portal separate from other users allowing for additional functionality. For example, the treating physician may provide for threshold values based on specific patient needs. If the patient requires an altered BG level deviating from standard based on their lifestyle or specific disease being monitored, threshold values may be provided.

Where the treating physician sees necessary, an alert system may be established. The treating physician creates threshold values specifically relating to particular patient treatment. Where real-time information transmitted to the database is identified to be outside of the predetermined threshold values, an alert may be provided whereby the database transmits the anomaly or outlier immediately to the treating physician and indicates the nature of the alert. For example, where the BG level exceeds the established threshold value, the database may autonomously transmit the alert to the treating physician and/or the patient to indicate a potential health risks. In this embodiment, the treating physician is constantly engaged with the system.

Where the treating physician requires constant engagement with the system, the database may transmit realtime information to remote devices under the control of treating physician. For example, the treating physician may possess a mobile device such as a cellular phone which wireless engages the database. Information is then transmitted over the one or more networks where the one or more networks are wireless networks.

Where information is received, options are provided through the GUI to allow of rapid access to further information and data or communication means between the physician and the patient.

The system may engage with a global positing system (GPS) operating on the devices being used by the physician or the patient. The GPS system can provide further information transmitted to the database to identify a geolocation of the user. Such geolocation information provides for preliminary consideration on downstream activities taken by the patient or physician.

The GPS information may also assist in the location of medical services nearby, should the need arise. For example, where the data transmitted through the one or more networks triggers an alarm, the severity of the alarm may be indicated and based on the severity of the resulting alarm due to the data being outside of the predetermined threshold values, instruction for the patient may be provided through a patient side GUI instructing the patient to seek medical attention. In an alternative embodiment, where the triggered alert or data value is sufficiently severe, the system may provide for autonomous contact of emergency services. While the autonomous nature of the contact may happen without verification, the patient and or physician receiving the alert may also be presented with a confirmation for transmitted communications from the system/device to emergency medical services.

In another embodiment, a device is provided having a BG analysis mechanism disposed therein whereby the device is physically connected to a mobile device connected to the one or more networks. For example a finger-stick device having a sample obtaining means to breach the patient's skin and obtain a sample of blood is integrated into a device which uptakes the sample and performs one or more analysis techniques on the sample allowing for an output value. The First device then transmits the output information, among other patient related information through a mobile device, to which it is attached, to the remote database. The information is then observable and analyzed in accordance with the method disclosed herein.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A system for remotely capturing and processing daily blood glucose data and of a patient comprising:
a. a first device configured to obtain the analyzed readings of glucose level data and in wireless communication with the Second device, transmit the analysis readings of glucose level in realtime, wherein the data is validated for at least a reading of blood glucose level, and wherein the blood glucose analysis data is wirelessly transmitted to the database on second device, wherein the database stores the analysis; and
b. a second device receiving the glucose analysis readings data, wherein the glucose analysis readings data is received in the second device from the first device, and wherein the analysis is combined with data from one or more analysis data of samples previously obtained.

2. The system of claim 1, wherein the patient is authenticated, wherein one or more unique medical record identifiers are provided, wherein the one or more unique medical record identifiers are associated with the patient, and wherein when the one or more unique medical record identifiers are accepted, a confirmation is received by the first device from the second device.

3. The system of claim 1, wherein the analyzed data of the sample is cross-referenced with an industry standard glycemic thresholds, wherein the glycemic status is predetermined based on the patient, and wherein a level of glycemic status is identified, wherein the glycemic level corresponds to the blood glucose concentration.

4. The system of claim 3, wherein if the level of glycemic status deviates from a predetermined threshold level, an alert is provided to at least the first device and the user interface system on the second device.

5. The system of claim 3, wherein a display provides visual representation of the analysis and the level of glycemic status.

6. The system of claim 1, wherein a user engages the first device, wherein the user inputs one or more data into the first device, wherein the one or more data includes at least a threshold value of the concentration of blood glucose.

7. The system of claim 5, wherein the visual representation comprises output patient glucose, insulin charts, advisory messages, and patient alerts based on the concentration of blood glucose.

8. The system of claim 3, wherein the database stores one or more medications prescribed to the patient.

9. The system of claim 1, wherein a physician module within the second device is configured to alter medication type and medication dosage.

10. The system of claim 1, further comprising a telecommunications module configured to communicate patient status data to an emergency responder.

11. The system of claim 10, further comprising a data provisioning module to upload data to a third party system.

12. The system of claim 1, wherein the first device is a cellular phone or any other mobile data communication device.
